(19) United States
(12) Patent Application Publication
(10) Pub. No.: US 2006/0259021 A1
(43) Pub. Date: Nov. 16, 2006

(54) Diode-laser-pumped ultraviolet and infrared lasers for ablation and coagulation of soft tissue

(76) Inventor: J. T. Lin, Oviedo, FL (US)

Correspondence Address: J. T. Lin, 4532 Old Carriage Trail, Oviedo, FL, 32765, US

(21) Appl. No.: 11/127,151

(22) Filed: May 12, 2005

(51) Int. Cl. A61B 18/18

(52) U.S. Cl. 606/4

(57) ABSTRACT

Method and systems for eye surgery for the treatment of presbyopia, ocular hypertension and glaucoma and other soft tissue surgeries are disclosed. System design parameters of lasing crystals (Nd:YAG, Nd:YLF, Er:YAG and Er:YSGG), nonlinear crystals (KTP, BBO, LBO), laser cavity configuration and energy delivery means are disclosed for diode-laser-pumped lasers with output wavelength at UV (263 or 266 nm), green (527 or 523 nm), and mid-IR (2.78 or 2.94 microns). Dual function of ablation and coagulation is proposed by a mode control means. The preferred diode-laser includes a wavelength at about 0.75 to 0.98 microns with power about 15 to 40 W and used in a side-pumping configuration to generate UV or IR having an energy per pulse about 3 to 30 mJ, power of about 0.1 to 1.5 W and operated at free running mode (IR laser) or pulsed mode (UV laser).

### BACKGROUND OF THE INVENTION 1. Field of the Invention

This invention relates to methods and systems for the treatment of presbyopia and glaucoma and for use in microsurgery of soft tissue of other parts of human body. This invention also relates to the generation of ultraviolet and infrared coherent light by diode-laser-pumped technology. 2. Prior Art

The first mini-size semiconductor diode pumped laser (DPL) was demonstrated in the early 1980 by Professor Yariv of Stanford University. Due to the available power of GaAs diode laser, the output of the mini-DPL was only few miliwatt (mW) at a near infrared (IR) wavelength (1064 nm). The recent development of high-power (larger than 20 W) diode array promotes the high-power DPL technology for practical applications, which, however, are limited to industrial and military. Low power DPL about (1 to 10) mW has been used for medical uses such as retina treatment of an eye, which however, are limited to a green spectrum (532 nm). Due to the technical difficulty and lack of new applications, DPL's in the UV spectrum of 0.2 to 0.4 microns have not been developed. The existing medically used DPL's, therefore, are limited to two spectra, the green (532 nm) and near IR (1064 nm). Solid-state UV lasers (at about 0.2 microns) for refractive surgery (cornea reshaping) were first disclosed by the present inventor in U.S. Pat. No. 5,144,630, which was also limited to a flash-lamp-pumped (FLP) technology. The available output power of DPL in UV spectrum about (210 to 360) nm is limited to less than about (0.1 to 0.5) W due to the low conversion efficiency of nonlinear crystals which convert the near IR to a green and then to a UV output. This multiple conversion procedure in UV DPL makes the overall efficiency (optical to optical) lower than about 2%.

The second area of ophthalmic application is the use of a mid-IR laser having a spectrum about (2.8 to 3.2) microns which was also disclosed by the present inventor in U.S. Pat. Nos. 5,520,679 (Lin-679) and 6,258,082 (Lin-082) for ophthalmic applications. Although DPL at UV (0.19 to 0.21) micron and Q-switched Er:YAG were mentioned in the prior arts of Lin (claims 2 of Lin-679 and claim 9 of Lin-082). There were no specific configurations disclosed for system design for any practical uses. Furthermore, the DPL UV laser disclosed in Lin-679 is limited to corneal reshaping which requires a UV wavelength of (193 to 213) nm, much shorter than the 265 nm disclosed in this invention, and it is very difficult to obtain the required power level for corneal reshaping. No system of DPL UV-213 or Er:YAG has been made since it was proposed over 10 years ago by the present inventor.

Similar to the earlier discussions for DPL in UV, the conversion efficiency of DPL in mid-IR is also limited to about (2%-3%) due to the lack of high-power diode array and the rather low emission efficiency in the laser crystals of Er:dopped, which is about 2 to 5 times lower than that of Nd-dopped crystals, such as Nd:YAG or Nd:YLF.

The method and design aspects for clinically useful DPL in the above-mentioned two preferred spectra, UV at about (210-370) nm and IR at about (2.8-3.2) microns, are not obvious. They involve the specific parameters of pumping diode laser, nonlinear and lasing crystals, optimal optical cavity design (to overcome the low efficiency) and delivery of the output laser beam. Depending on the types of lasing crystals (Nd or Er dopped), the preferred diode laser spectrum of this invention includes about 0.81 micron for DPL Nd:YAG, Nd:YVO4, or Nd:YLF for UV output; and about 0.75 to 0.98 microns for DPL Er:YAG, Er:YSGG, Er:Cr:YSGG, Er:Cr:Th:YAG or Er:YALO3 for IR output.

Another preferred cavity configuration of this invention includes the use of high-power diode array and side-pumping. Given an overall efficiency of about 1% to 3%, the pumping diode laser power shall be about 10 W to 40 W in order to obtain the required output of UV or IR lasers power of about 0.1 to 1.5 W for microsurgeries of soft tissues of an eye or other preferred parts of human body, including, but not limited to, soft tissue in the mouth, ear, head and neural-systems.

Mid-IR laser of Er:Cr:YSGG (at 2780 nm) was disclosed in the prior arts of U.S. Pat. Nos. 5,342,198; 6,086,367; 6,567,582, however, it used a standard commercially available flash-lamp-pumped (FLP) system and was limited to dental applications, where much higher power of about 1 to 6 W is required. The power range of 0.1 to 1.5 W disclosed in this invention for soft tissue microsurgeries is much lower than that of FLP. Therefore, the preferred DPL in mid-IR of this invention is for small scale ablation, cutting, hemostats, or coagulation, about 0.1 to 1.0 cm in length. In addition, the above prior arts for dental uses requires laser energy per pulse about 100 to 300 mJ, much higher than what can be produced from the current technology of DPL, less than about 30 mJ.

None of the above disclosed specific information has been disclosed in prior arts and, to my best knowledge, there are no commercially available DPL systems at the two preferred spectra of UV and IR disclosed in this invention which also discloses the new medical uses of these DPL's.

We also note that Lin-679 proposed DPL at UV about 213 nm required for corneal reshaping (a wavelength comparable to that of an ArF excimer at 193 nm), however, is not required in our new procedure for presbyopia treatment, which only requires a UV at about 263 or 266 nm. The UV-266 generated from the fourth harmonic of Nd:YAG is about 5 to 10 times more efficient than the UV-213 (fifth harmonic). Therefore, system of UV-266 would be more practical and technically achievable than UV-213 which is not yet available using current DPL technology, although it was proposed by Lin-679 which is the CIP of Ser. No. 985,617 (in 1992), over 10 years ago.

The current technology is available only for UV-213 in FLP, not in DPL, which may need another 10 to 15 years for a clinically useful system to be developed. In comparison, the DPL at UV-266 could be made to meet clinical requirements based on the teaching of the present inventions, but not by the teaching of prior arts of Lin's or others.

Based on above background and prior arts discussions, one objective of the present invention is to provide systems and methods to obviate the drawbacks of prior arts and disclose new applications.

It is yet another objective of the present invention to disclose specific parameters of the pumping laser, the nonlinear and lasing material and cavity configurations to overcome the rather low efficiency of DPL's at the specified UV and IR spectrum.

It is yet another objective of the present invention to disclose the UV and IR laser energy, power and spot size required for efficient ablation of the sclera or ciliary-body for the treatment of presyopia and glaucoma.

It is yet another objective of the present invention to disclose the means of laser energy delivery.

It is yet another objective of the present invention to disclose the efficiency analysis at a given pumping of power for both UV and IR lasers.

It is yet another objective of the present invention to disclose the structure and features of an integrated system based on specific parameters and configuration designs.

It is yet another objective of the present invention to disclose the clinical aspects of the proposed medical procedures, where the new concept based on the ablation threshold is introduced.

It is yet another objective of the present invention to disclose the ablation and coagulation dual-function in one single-laser for applications related to soft tissue surgery.

Further objectives of this invention will become apparent from the description of this invention to be detailed as follows.

### SUMMARY OF THE INVENTION

System configuration is disclosed with detailed specifications of each of the elements including pumping diode laser, lasing crystals, nonlinear crystals and optics. DPL with dual wavelength at green and at UV can be selected for ablation or coagulation. The preferred embodiments also include delivery means of articulated arm or optical fiber. Another preferred design includes a handheld configuration having the laser cavity and optics integrated to a dimension about 1.5×2.0×20 cm.

Given an overall efficiency of about 1% to 2% for UV laser at 266 or 263 nm from Nd:YAG or Nd:YLF, and about 3% to 4% for IR laser at 2.78 um (micron) from Er:Cr:YSGG or 2.94 um from Er:YAG, the required pumping diode laser power is about 5 W to 40 W and wavelength of about 809, 750 to 980 nm, where a side-pumping configuration is preferred.

Clinically tested parameters of ablation threshold energy (E*) or power density (I*) are used to specify the required energy, power, spot size and pulse duration of both UV and IR laser, where the preferred ablation rate is based on a power level about 2 to 3 times of I*, which is about 150 to 250 MW/cm/cm (for UV laser) and about 5 to 10 KW/cm/cm (for IR laser).

Another preferred embodiment includes a laser energy per pulse (E) for efficient ablation is about 3 to 15 mJ (for UV laser) and about 10 to 40 mJ (for IR laser) for general microsurgeries; and most preferable about 6 to 8 mJ (for UV) and 15 to 20 mJ (for IR) for the treatment of presbyopia or glaucoma by soft tissue ablation of an eye.

The preferred energy for coagulation of soft tissue is less than about 3 mJ and 10 mJ, for UV and IR laser, respectively, with a spot size of about 0.5 to 1.0 mm (at the treated tissue surface).

The soft tissue may include eye, skin, muscle, heart, liver, mucosa, gingival, kidney, brain and vessels for medical applications including vision correction, dental, oral or auditory treatment, neuroendoscopic surgery, laparoscopic, liposuction and arthroscopic, etc.

### BRIEF DESCRIPTION OF THE DRAWINGS

FIG. 1. System schemes show the structure of a diode-pumped laser (DPL) cavity and nonlinear crystals for the generation of green and UV laser.

FIG. 2. Schemes show a handheld structure (A), and coupled to an articulated arm (B) for a DPL with UV output.

FIG. 3. Schemes show a DPL with IR output coupled to an optical fiber (A), structure of the hand piece (B) and a handheld structure (C).

FIG. 4 Diagram shows partial structure of human eye and the location of laser treated area.

### DETAILED DESCRIPTION OF THE INVENTION AND THE PREFERRED EMBODIMENTS

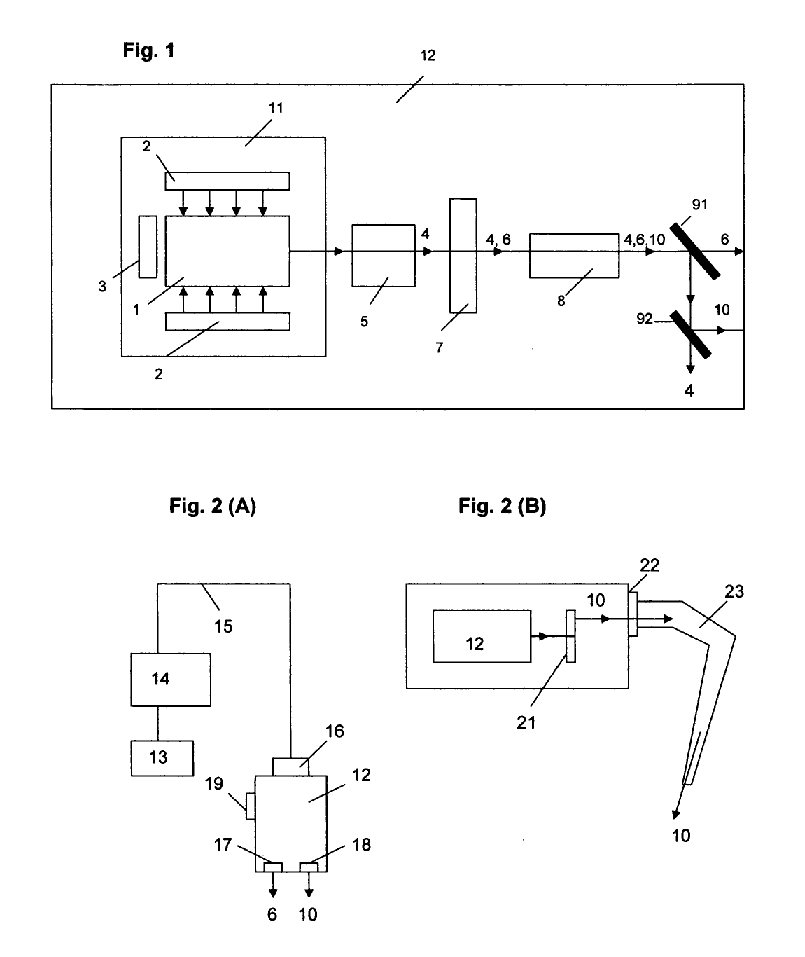

As shown in FIG. 1 (A), a lasing crystal 1 is side-pumped by a pair of diode-laser array 2 and reflected by a back optics 3 to produce an output beam 4 which is mode controlled by a Q-switch device 5 for pulse output. The fundamental beam 4 having an IR wavelength at about 1064 nm or 1053 nm, for lasing crystal Nd:YAG or Nd:YLF, respectively, is converted to a green output 6 (at 532 or 527 nm) via a second harmonic generation nonlinear crystal (SHC) 7 which is further converted to a UV output 10 (at about 266 or 263 nm) via a fourth-harmonic generation crystal (FHC) 8 which may be separated from the IR and green by a pair of dichromatic optics 91 and 92. We also define the laser cavity 11 and the integrated unit 12.

The preferred specifications of materials used in FIG. 1 include: (1) back surface of the lasing crystal 1 is anti-reflection (AR) coated at the fundamental wavelength (IR), and its front (output) surface is partially reflecting (about 2% to 10%) to the IR; (2) the diode laser array 2 has a wavelength at about 809 nm and has a length comparable to the lasing crystal about 5 to 20 mm; (3) the Q-switch 5 is a standard electro-optics (EO) switch having both end surfaces AR-coated at IR wavelength (1053 or 1064 nm); (4) the SHC includes KTP, KDP, CDA, CBO, LBO or BBO, with most preferable of KTP; with both surfaces AR-coated at both IR and green and having a length about 2 to 10 mm and end surface area larger than that of the lasing crystal, about 4×4 mm; (5) the FHC includes BBO, LBO, KDP or any other UV transparent crystals having a phase matching angle, both surfaces AR-coated at green and UV, where both FHC and SHC preferred to be in a heated housing for stable output power; (6) the dichromatic optics 91 and 92 having 45 degree in angle with respect to the incident laser beams, shall have a front surface high reflection (HR) coated at UV and high-transparent (HT) at green and IR.

The above described system specifications are important in order to achieve maximized UV output power in a DPL system which, unlike FLP lasers, is limited by the available power of the pumping diode. Another preferred embodiment of this invention shown by FIG. 1 includes the dual wavelength output at UV 10 and green 6 from one laser unit which allows us to conduct a dual function of ablation (using UV) and coagulation (using green or UV). Greater detail will be disclosed in FIG. 2. There is no commercial system developed, either DPL or FLP, for a dual medical applications as disclosed in this invention.

FIG. 2 (A) shows another preferred embodiment, where a computer 13 is used to control the power supply 14 connected by a long power line 15 (about 0.5 to 1.5 meter) and a connector 16 to the handheld unit 12 which is further detailed in FIG. 1 including a laser cavity 11 and other components to produce output at UV 10 and green 6. The selection of UV or green is further controlled by an attached switch 19 which can mechanically or electronically open one of the two shutters 17 and 18. This mode switch allows us to conduct ablation mode (AM) and coagulation mode (CM) in soft tissue surgeries where bleeding shall be minimized. Another preferred embodiment includes means of changing the UV laser spot size, where larger laser spot (at the treated surface) serves as a CM, whereas a small spot as an AM. For a given UV laser energy (E), the spot size expansion may be done by changing the focusing length of the optics or manually controlling the distance between the laser focusing length of the optics or manually controlling the distance between the laser focusing point and the treated soft tissue surface. Mode switch from AM to CM can also be done by reducing the input power (or voltage), such that the output UV power is below its ablation threshold, which is about (1.0-3.0) mJ for a typical nanosecond laser. Yet another preferred embodiment includes tuning the FHC angle away from its phase matching director, such that only the green is generated for CM.

FIG. 2(B) shows another preferred embodiment including the UV output beam 10 is delivered to the treated area by an articulated arm 23, which is connected to the laser cavity unit 12 by a connector 22 and aligned by an optics unit 21 having a pair of 45 degree HR mirror at UV. Because of the poor transparence at UV of about 265 nm in most existing optical fiber, the above described preferred embodiments shown in FIG. 2 (A) and (B) are particularly attractive. These systems are not currently available, particularly in DPL and for medical uses, although a FLP UV laser coupled to an articulated arm were disclosed by Lin (U.S. application Ser. No. 11/008,108).

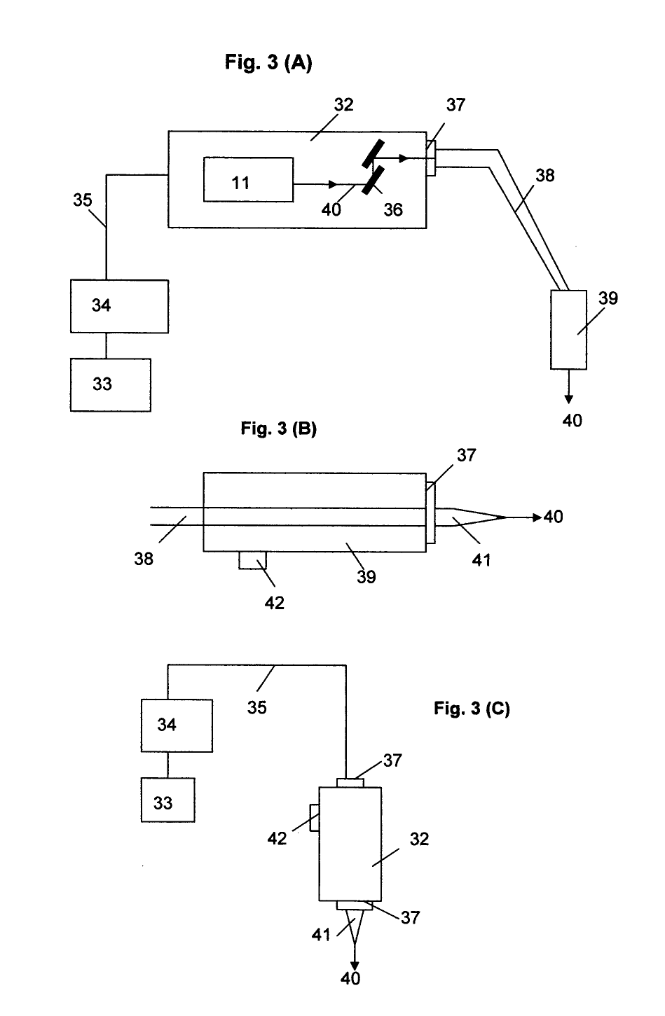

FIG. 3 (A) to 3 (C) show the preferred embodiment for an IR laser, where the laser cavity 11 has the same structure as that of UV laser in FIG. 1, except the following parameters are changed: the lasing crystal is Er:YAG or Er:YSGG; the pumping diode laser is about 750 to 980 nm in wavelength; the coating specifications are the same as that of UV laser, except the IR output 10 of the system becomes 2.94 or 2.78 microns (rather than 1064 or 1053 nm). In addition, the IR output 10 is directly from the laser cavity 11 without using nonlinear crystals. As shown in FIG. 3(A), a computer 33 is used to control the power supply 34 which is connected to the laser unit 32 by a wire 35. The laser unit 32 consists of the laser cavity 11 and a pair of HR coated (at IR of 2.78 or 2.94 micron) mirror to align the output beam 11 and coupled to an optical fiber 38 which is connected by 37 to the unit 32 and has a hand piece 39 to deliver laser beam 40 to the treated area. Detail of the hand piece 39 is further shown in FIG. 3(B), consisting of an output connector 37 such that the end-piece 41 can be detached from 39 for multiple uses or being used as disposable.

Another preferred embodiment is shown by FIG. 3(C), where the laser unit 32 itself becomes the hand piece without the need of an optical fiber 38 to deliver the IR laser. IR optical fiber suffers problems of losing laser power and damage. The preferred configuration of FIG. 3 (C) obviates these drawbacks. Also shown in FIG. 3 (B) and (C)is a preferred switch 42 attached to the hand piece 39 or 32, which allows the surgeon to select AM or CM. The mode switch may be also attached to the footswitch as an alternatives.

The preferred means of mode control of AM and CM for dual-function uses shall also includes (for both UV and IR lasers): (1) laser energy\pulse control (from low to high); (2) laser fluency control, by changing laser beam spot size (from small to big spot), where the fluency is defined by energy/spot area); and (3) laser emission model control (from continuous mode to a pulsed mode, such that the peak power density increases).

One preferred example is an Nd:YAG lasers with UV (355 nm or 266, or 215 nm) outputs and can be switched from low to high power mode, or from continuous-wave (CW) to Q-switched model. The second preferred example is to use a mid IR laser (2.7 to 3.2) um operated at free running (about few hundreds of microseconds pulse duration) or CW mode and can be switched for its power level from about (0.1 to 0.2) W to about (0.3 to 5 W) or switching its spot size from about (0.8 to 1.5) mm to (0.2 to 0.7) mm. We shall note that the spot size change (reduced) of 30% produce a power density (or fluency) of 69% more which allows us to control the laser mode from CM to AM. In addition, the peak power may increase a factor of 100 by switching from a long pulse (say 1,000 usec) to a short pulse (say 100 usec) mode.

We have tested an Er:YAG laser (at 2.94 um, pulse width at about 200 usec) and a diode laser at about 1.5 um (CW mode) at low power and high power levels by the proposed means and confirmed the control/switch of coagulation/thermal mode (CM) and ablation model (AM) on animal eyes. The CM showed some kind of thermally burned “white” spot whereas the AM showed no thermal damage/color with “sharp” ablating edges. The threshold energy/pulse (for spot of 0.6 mm) was about 10 mJ in Er:YAG laser and threshold power was about 0.3 W (spot of about 0.1 mm) in diode laser at 1.5 um.

We shall now analyze the efficiency of DPL, for both UV and IR laser and the ablation threshold as follows. These analyses together with the specifications of FIG. 1 to 3 are the critical elements of system design and they have not been disclosed in prior arts. Without knowing these detailed parameters and teaching, a DPL would not produce the required energy or power for the medical procedures proposed in this invention. This is also part of the reasons why there is no commercial DPL systems developed for the medical surgeries proposed in the present invention, over 10 years after the present inventor proposed the concept of DPL for medical uses in 1992. One of the key issues of DPL technology is how to improve the overall efficiency which is governed by the material property of the lasing crystal, the available pumping laser power at a particular spectrum matching the absorption band of the lasing crystal, and the laser cavity design, including all the coating specifications of the elements. FIG. 1-3 described most of the above aspects, yet extra analyses are still needed as follows:

First, we introduce the concept of “ablation threshold” defined by the power density I=E/(AT), where E is laser energy per pulse, T is laser pulse width and A is the area of beam spot (at the treated surface). Using the commercial flash-lamp-pumped (FLP) lasers at UV (266 nm) and at 2.94 microns (Er:YAG), we had tested the following clinical parameters. For a UV laser, with T about 5 ns and spot size (on tissue surface) about 0.6 mm, we found that laser energy (E) about 6 mJ is required for a reasonable ablation rate (B), whereas coagulation occurs (the CM) when E is lower than about 2 to 3 mJ. For an IR laser (at about 2.94 or 2.8 microns), our testing on cadaver eye shows that higher energy than UV laser, about 15 mJ is needed for ablation due to the longer T, about 500 microsecond, than that of UV laser, and ablation threshold is about 5 to 10 mJ (at spot size about 0.7 mm). Therefore, we calculated the threshold power density (I*) to be about 150 to 250 (MW/cm/cm) for UV laser, and much lower, about 5 to 10 (KW/cm/cm) in IR laser. This difference of I* in UV and IR laser is partially due to their absorption difference, where UV laser energy absorbed by protein in tissue, and IR mainly by the water in tissue which is much stronger and lower I* is expected.

Using the above requirements for efficient ablation, with an energy (or power density) preferred to be about 2 to 3 times of the threshold value, we then may analyze the required parameters in DPL systems as follows.

For UV laser, the preferred pumping diode power about 20 W will produce fundamental output (at 1064 or 1053 nm) about 4 to 6 W (given an optical-to-optical efficiency of 20% to 30%). Due to the excellent beam quality in DPL, we expect about 60% to 70% efficiency in converting to green power about 3 to 4 W, which shall be further converted to generate UV output at about 0.6 to 0.8 W (given a 20% efficiency). Therefore, the overall efficiency is about 3% to 4% which, however, must be discounted to half, or about 1.5% to 2% if one to include the loss due to EO Q-switch is included. So, the preferred pumping power of this invention includes about 5 to 40 W to produce a UV (at 266 or 263 nm) power about 0.1 to 0.8 W. This average power shall meet our clinical needs, if the UV laser is operated at short pulse duration of about 5 to 200 ns with energy per pulse of about 3 to 15 mJ. For longer pulse, however, we shall need higher UV energy or power, since I=E/(TA) as discussed earlier. For eye surgery applications disclosed in this invention, the most preferable UV power is about 0.15 to 0.3 W which only needs diode laser power of about 7.5 to 15 W.

For IR lasers (at about 2.7 to 2.94 microns), the pumping efficiency (from diode to IR output) is much lower than that of Nd:YAG or Nd:YLF, due to the energy level structure of the lasing crystals Er:YAG and Er:YSGG. Using an estimated efficiency of about 2% to 3% (about 10 times lower than Nd:YAG), the Er-dopped YAG or YSGG shall produce about 0.4 to 0.6 W of IR output with an input diode laser power about 20 W. Unlike the Q-switched UV laser, free running mode with pulse width about 100 to 700 microsecond (usec), comparable to FLP system, will be acceptable for the soft tissue ablation. We note that Q-switched short pulse in UV system is required in order to achieve efficiency in nonlinear crystals, particularly the FHC. Free running mode is preferred in IR laser, however, a short pulse, say 100 to 200 usec is the most preferable range, because a longer pulse of 500 usec has a power density only about 20% of a 100 usec laser, and higher energy per pulse would be needed. For microsurgeries need of about 0.3 to 0.8 W of IR power, the preferred pumping diode power is about 15 W to 40 W. For eye surgery, the most preferable range of IR laser power is about 0.2 to 0.5 W and energy per pulse of about 10 to 25 mJ, which can be generated by a diode power of about 15 to 30 W. For dental or other surgeries, these preferred amounts in eye surgery would be higher, particularly for ablation length longer than 5 mm.

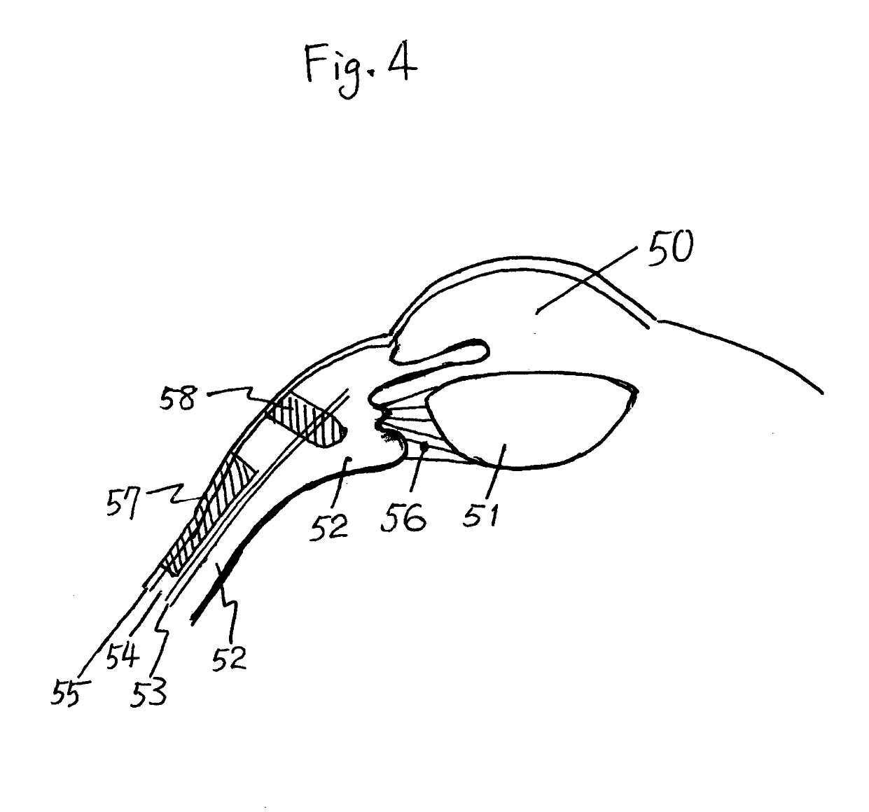

Another preferred embodiment is related to clinical or surgical techniques. As shown in FIG. 4, the accommodation of human eye 50 to see near and far is governed by the axial movement of the lens 51 and its surface curvature change, resulted from the contraction of the ciliary-body (CB) 52 which is beneath the choroids layer 53, sclera 54 and conjunctiva 55. In the prior arts of Lin (U.S. Pat. Nos. 6,263,879; 6,258,082, “Lin-79-82), flash-lamp-pumped (FLP) lasers were used to remove a portion of the sclera tissue to improve accommodation of presbyopia. These FLP lasers are bulky and hard to maintain. DPL offers the advantages of compact, long life-time and much easier to operate and maintain. The handheld designs in FIG. 2 (A) and FIG. 3 (C) are another unique feature of DPL, where the laser unit (laser cavity and optics) may be integrated to a preferred dimension about 2.0×4.0×20 cm or most preferable about 1.5×2.0×20 cm. In addition to the technical advantages, we also disclose a new method of surgery for clinical advantage as follows.

Yet referring to FIG. 4, prior arts of Lin-79-82 remove a portion of the sclera layer 57, which is superficial and “remote” to the zonules 56 and lens 51. Therefore, Lin's prior arts clinically suffer low efficacy and postoperative regression. With the present new method, much deeper tissue layer within the CB (shown by 58) is removed, therefore, higher efficacy and less regression are expected. Furthermore, the risk of perforation in Lin-79-82 is not a concern in the present new method, because the layer 58 has a total thickness about 1.5 to 2.0 mm comparing to the layer 57 only about 0.5 to 0.6 mm. Therefore, another preferred embodiment of this invention includes the removal of a portion of the CB tissue, with or without the removal of conjunctiva or sclera layer, having an ablation depth about 10% to 50% of the CB thickness (about 1 to 1.5 mm). It is yet another preferred method that the laser ablation shall be along the direction and within the area where CB has a maximal thickness to avoid perforation. The preferred total ablation depth is about 0.4 to 1.4 mm, and most preferable of about 0.6 to 1.2 mm. The preferred applications of laser ablation a portion of CB, include the increase of accommodation for presbyopic patients, decrease the intraocular pressure for hypertension and treat primary open angle glaucoma.

Another preferred clinical use of DPL includes soft tissue ablation, cutting, coagulation relating to dental, oral, auditory treatment of human body and neural system surgeries.

The invention having now been fully described, it should be understood that it may be embodied in other specific forms or variations without departing from the spirit or essential characteristics of the present invention. Accordingly, the embodiments described herein are to be considered to be illustrative and not restrictive.

I claim:

1. A method for treating eye disorder of presbyopia and glaucoma by ablating the soft tissue of an eye, comprising the steps of:
(a) selecting a laser beam having a predetermined energy, spot size and wavelength;
(b) selecting a beam delivery means which delivers said laser beam energy to a predetermined area with a predetermined pattern of an eye;
whereby the treated eye will have increased accommodation or decreased intraocular pressure.

2. A surgical method of claim 1, wherein said laser beam is an ultraviolet laser having a wavelength range of about 263 or 266 nm and a pulse energy of between about 3 to 15 mJ and power of between about 0.15 to 0.3 W on said soft tissue.

3. A surgical method of claim 1, wherein said laser beam is a diode-laser-pumped Nd:YAG, Nd:YVO4, or Nd:YLF laser and frequency converted by harmonic generation nonlinear crystals of KTP, KDP, CDA, CBO, BBO or LBO, having an overall conversion efficiency between about 1% and 2%.

4. A surgical method of claim 3, wherein said diode-laser is a semiconductor diode array having a wavelength about 809 nm and power of between about 7.5 and 15 W and used in a side-pumping configuration having an optical-to-optical efficiency between about 20% and 30%.

5. A surgical method of claim 4, wherein said side-pumping configuration generates ultraviolet (about 265 nm) laser beam.

6. A surgical method of claim 5, wherein said UV or green laser is selected for the ablation or coagulation of said soft tissue by a pair of dichromatic optics and shutters.

7. A surgical method of claim 1, wherein said delivery means includes an articulated arm which delivers said laser beam at ultraviolet wavelength of about 263 or 266 nm to said soft tissue.

8. A surgical method of claim 1, wherein said beam delivery means includes a handheld piece having a dimension about 1.5×2.0×20 cm and containing the laser cavity and optics.

9. A surgical method of claim 1, wherein said laser beam is a diode-laser pumped infrared laser of Er:YAG, Er:YSGG, Er:Cr:YSGG, Er:Cr:Th:YAG or Er:YALO3 having an output wavelength of about 2.7 to 2.94 microns, energy per pulse between about 5 and 30 mJ and output power between about 0.2 and 0.5 W.

10. A surgical method of claim 3, wherein said diode-laser is a semiconductor diode array having a wavelength about 750 to 980 nm and power of between about 15 and 30 W and used in a side-pumping configuration having an optical-to-optical efficiency between about 2% and 3%.

11. A surgical method of claim 1, wherein said predetermined pattern includes radial lines, curves, ring-dot or non-specific patterns around the area outside the limbus.

12. A surgical method of claim 1, wherein said soft tissue includes the conjunctiva layer, sclera tissue or ciliary body of an eye.

13. A surgical method of claim 1, wherein said soft tissue is ablated by said laser beam to a depth of between about 0.4 and 1.4 mm, most preferable between about 0.6 and 1.2 mm, including removal of about 10% to 50% of the ciliary body thickness with or without removal of the conjunctiva or sclera tissue.

14. A surgical method of claim 9, wherein said infrared laser energy is reduced by a mode control means to a value below the ablation threshold for coagulation of said soft tissue.

15. A surgical method of claim 14, wherein said mode control means includes changing the pulse duration or energy per pulse of the said laser beam, or changing the spot size of said laser beam at the treated said soft tissue surface.

16. A surgical method of claim 14, wherein said ablation threshold energy is between about 5 and 10 mJ per pulse at a said laser beam spot size of about 0.7 mm.

17. A method for ablation or coagulation of soft tissue of human body for vision, dental, oral or auditory treatment or other microsurgeries, comprising the step of:
(1) selecting a laser beam having a predetermined energy, spot size and wavelength;
(2) selecting a beam delivery means which delivers said laser beam energy to the treated said soft tissue.

18. A surgical method of claim 17, wherein said laser beam is a diode-laser-pumped ultraviolet laser having a wavelength of about 263 or 266 nm or infrared laser having a wavelength of about 2.7 to 2.94 micron having an output power of between about 0.1 and 1.5 W, energy per pulse of between about 3 and 30 mJ.

19. A surgical method of claim 18, wherein said diode laser has a wavelength about 0.81 or 0.75 to 0.98 microns and power of between about 10 and 40 W.

20. A system for treating presbyopia, glaucoma and other soft tissue surgeries, the system comprising:
(a) a laser beam having a predetermined energy, spot size and wavelength.
(b) a beam delivery means to deliver said laser beam energy to a predetermined pattern and area of an eye or other parts of human body.

21. A system of claim 20, wherein said laser beam is an ultraviolet laser having a wavelength range of about 263 or 266 nm and a pulse energy of between about 3 to 15 mJ and power of between about 0.1 to 1.5 W on said soft tissue.

22. A system of claim 20, wherein said laser beam is a diodelaser-pumped Nd:YAG, Nd:YVO4 or Nd:YLF laser and frequency converted by harmonic generation nonlinear crystals of KTP, BBO or LBO, having an overall conversion efficiency between about 1% and 2%.

23. A system of claim 22, wherein said diode-laser is a semiconductor diode array having a wavelength about 809 nm and power of between about 5 and 40 W and used in a side-pumping configuration having an optical-to-optical efficiency between about 20% and 30%.

24. A system of claim 23, wherein said side-pumping configuration generates both ultraviolet (UV) about 266 nm, and green (about 532 nm) laser beam which are separated by a 45 degree angle dichromatic optics.

25. A system of claim 24, wherein said UV or green laser is selected for the ablation or coagulation of said soft tissue by a pair of dichromatic optics and shutters.

26. A system of claim 20, wherein said delivery means includes articulated arm which delivers said laser beam at ultraviolet wavelength of about 263 and 266 nm to said soft tissue.

27. A system of claim 20, wherein said beam delivery means includes a handheld piece having a dimension about 1.5×2.0×20 cm and containing the laser cavity and optics.

28. A system of claim 20, wherein said laser beam is a diode-laser pumped infrared laser of Er:YAG, Er:YSGG. Er:Cr:YSGG, Er:Cr:Th:YAG or Er:YALO3 having an output wavelength of about 2.7 to 2.94 microns, energy per pulse between about 5 and 30 mJ and output power between about 0.1 and 1.5 W.

29. A system of claim 28, wherein said diode-laser is a semiconductor diode array having a wavelength about 750 to 980 nm and power of power of between about 5 and 40 W and used in a side-pumping configuration having an optical-to-optical efficiency between about 2% and 3%.

30. A system of claim 20, wherein said predetermined pattern includes radial lines, curves, ring-dot or non-specific patterns around the area outside the limbus.

31. A system of claim 20, wherein said soft tissue includes the conjunctiva layer, sclera tissue, or ciliary body of an eye.

32. A system of claim 20, wherein said soft tissue is ablated by said laser beam to a depth of between 0.4 and 1.4 mm, most preferable between about 0.6 and 1.2 mm, including removal of about 10% to 50% of the ciliary body thickness with or without removal of the conjunctiva or sclera tissue.

33. A system of claim 28, wherein said infrared laser energy is reduced by a mode control means to a value below the ablation threshold for coagulation of said soft tissue.

34. A system of claim 33, wherein said mode control means includes changing the pulse duration or energy per pulse of the said laser beam, or changing the spot size of said laser beam at the treated soft tissue surface.

35. A system of claim 33, wherein said ablation threshold energy is about 5 to 10 mJ per pulse.
